(19) United States
(12) Patent Application Publication
(10) Pub. No.: US 2005/0107875 A1
(43) Pub. Date: May 19, 2005

(54) Accommodating lens with haptics

(75) Inventor: J. Stuart Cumming, Laguna Beach, CA (US)

Correspondence Address: ORRICK, HERRINGTON & SUTCLIFFE, LLP, 4 PARK PLAZA, SUITE 1600, IRVINE, CA, 92614-2558, US

(73) Assignee: eyeonics, inc.

(21) Appl. No.: 10/994,045

(22) Filed: Nov. 18, 2004

Related U.S. Application Data
(63) Continuation-in-part of application No. 10/454,280, filed on Jun. 3, 2003.
Application No. 10/454,280 is a continuation of application No. 10/057,691, filed on Jan. 24, 2002, now Pat. No. 6,638,306.
Application No. 10/057,691 is a division of application No. 08/858,978, filed on May 20, 1997, now Pat. No. 6,387,126.

(51) Int. Cl. A61F 2/16

(52) U.S. Cl. 623/6.37; 623/6.44; 623/6.51

(57) ABSTRACT

A flexible accommodating intraocular lens having anteriorly and posteriorly movable extended portions, such as T-shaped haptics, extending from a central solid biconvex optic to be implanted within a natural capsular bag of a human eye with the extended portions positioned between an anterior capsular rim and a posterior capsule of the bag, whereby during a post-operative healing period, fibrosis occurs about the extended portions to fixate the lens in the bag in a manner such that subsequent natural contraction and relaxation of the ciliary muscle moves and deforms the optic to provide vision accommodation.

This application is a continuation-in-part of application Ser. No. 10/454,280 filed Jun. 3, 2003, which is a continuation of application Ser. No. 10/057,691 filed Jan. 24, 2002, now U.S. Pat. No. 6,638,306, which is a divisional of application Ser. No. 08/858,978 filed May 20, 1997, now U.S. Pat. No. 6,387,126.

### BACKGROUND OF THE INVENTION

This invention relates generally to intraocular lenses to be implanted within a natural capsular bag in the human eye formed by evacuation of the crystalline matrix from the natural lens of the eye through an anterior capsulotomy in the lens. The invention relates more particularly to novel accommodating intraocular lenses of this kind having improved features including an optic and T-shaped haptics.

The human eye has an anterior chamber between the cornea and iris, a posterior chamber behind the iris containing a crystalline lens, a vitreous chamber behind the lens containing vitreous humor, and a retina at the rear of the vitreous chamber. The crystalline lens of a normal human eye has a lens capsule attached about its periphery to the ciliary muscle of the eye by zonules and containing a crystalline lens matrix. This lens capsule has elastic optically clear anterior and posterior membrane-like walls commonly referred to by ophthalmologists as anterior and posterior capsules, respectively. Between the iris and the ciliary muscle is an annular crevice-like space called the ciliary sulcus.

The human eye in patients under the age of 45 years possesses natural accommodation capability. Natural accommodation capability involves relaxation and contraction of the ciliary muscle of the eye by the brain to provide the eye with near and distant vision. This ciliary muscle action is automatic and shapes the natural crystalline lens to the appropriate optical configuration for focusing on the retina the light rays entering the eye from the scene being viewed.

The human eye is subject to a variety of disorders which degrade or totally destroy the ability of the eye to function properly. One of the more common of these disorders involves progressive clouding of the natural crystalline lens matrix resulting in the formation of what is referred to as a cataract. It is now common practice to correct a cataract by surgically removing the cataractous human crystalline lens and implanting an artificial intraocular lens in the eye to replace the natural lens. The prior art is replete with a vast assortment of intraocular lenses for this purpose.

Intraocular lenses differ widely in their physical appearance and arrangement. The present invention is concerned with intraocular lenses of the kind having a central optical region or optic, and portions which extend outward from the optic and engage the interior of the eye in such a way as to support the optic on the axis of the eye.

Intraocular lenses also differ with respect to their accommodation capability and their placement in the eye. Accommodation is the ability of an intraocular lens to accommodate, that is, to focus the eye for near and distant vision. Certain patents describe alleged accommodating intraocular lenses. Other patents describe non-accommodating intraocular lenses. Most non-accommodating lenses have immobile single focus optics which focus the eye at a certain fixed distance only and require the wearing of eye glasses to change the focus. Other non-accommodating lenses have bifocal optics which simultaneously image both near and distant objects on the retina of the eye. The brain selects the appropriate image and suppresses the other image, so that a bifocal intraocular lens provides both near vision and distant vision sight without eyeglasses. Bifocal intraocular lenses, however, suffer from the disadvantage that each bifocal image represents only about 40% of the available light, and a remaining 20% of the light is lost in scatter.

There are four possible placements of an intraocular lens within the eye. These are (a) in the anterior chamber, (b) in the posterior chamber, (c) in the capsular bag, and (d) in the vitreous chamber. The intraocular lens disclosed herein is for placement in the capsular bag.

### SUMMARY OF THE INVENTION

The present invention relates to accommodating intraocular lenses having a central optic and T-shaped haptics, and having improved accommodation.

This invention provides an improved accommodating intraocular lens to be implanted within a capsular bag of a human eye which remains intact within the eye after removal of the crystalline lens matrix from the natural lens of the eye through an anterior capsule opening in the natural lens. An improved accommodating intraocular lens according to the invention includes a central optic having normally anterior and posterior sides and extended portions spaced circumferentially about and extending generally radially out from the edge of the optic. These extended portions have inner ends joined to the optic and opposite outer ends movable anteriorly and posteriorly relative to the optic. To this end, the extended portions may be either pivotally or flexibly hinged at their inner ends to the optic or are resiliently bendable throughout their length. The terms “flex”, “flexing”, “flexible”, and the like are used in a broad sense to cover both flexibly hinged and resiliently bendable extended portions. The terms “hinge”, “hinged”, “hinging”, and the like are used in a broad sense to cover both pivotally and flexibly hinged extended portions.

The lens is surgically implanted within the evacuated capsular bag of a patient's eye through the anterior capsule opening in the bag and in a position wherein the lens optic is aligned with the opening, and the outer ends of the lens extended portions are situated within the outer perimeter or cul-de-sac of the bag. The lens has a radial dimension from the outer end of each extended portion to the axis of the lens optic such that when the lens is implanted within the capsular bag, the outer ends of the extended portions engage the inner perimetrical wall of the bag without unnecessarily stretching the bag.

As is known, after surgical implantation of the accommodating intraocular lens in the capsular bag of the eye, active endodermal cells on the posterior side of the anterior capsule rim of the bag cause fibrosis with shrinkage of the bag and fusion of the rim to the elastic posterior capsule of the bag. This fibrosis occurs about the lens extended portions in such a way that these extended portions and the lens are effectively “shrink-wrapped” by the fibrous tissue in such a way as to form radial pockets in the fibrous tissue which contain the extended portions with their outer ends positioned within the outer cul-de-sac of the capsular bag. The lens is thereby fixated within the capsular bag with the lens optic aligned with the anterior capsule opening in the bag. The anterior capsule rim shrinks during fibrosis, and this shrinkage combined with shrink-wrapping of the extended portions causes some radial compression of the lens in a manner which tends to move the lens optic relative to the outer ends of the extended portions posteriorly along the axis of the eye. The fibrosed, leather-like anterior capsule rim prevents anterior movement of the optic and urges the optic rearwardly during fibrosis. Accordingly, fibrosis induced movement of the optic occurs posteriorly to a distant vision position in which either or both the optic and the inner ends of the extended portions press rearwardly against the elastic posterior capsule of the capsular bag and stretch this posterior capsule rearwardly.

During surgery, the ciliary muscle of the eye is paralyzed with a ciliary muscle relaxant, i.e. a cycloplegic, to place the muscle in its relaxed state. Following surgery, a ciliary muscle relaxant, a cycloplegic, is introduced into the eye to paralyze the ciliary muscle throughout the post-operative fibrosis and healing period (from two to three weeks) to maintain the ciliary muscle in its relaxed state until fibrosis is complete. This drug-induced relaxation of the ciliary muscle prevents contraction of the ciliary muscle and immobilizes the capsular bag during fibrosis. By this means, the lens optic is fixed during fibrosis in its distant vision position within the eye relative to the retina wherein the lens presses rearwardly against and thereby posteriorly stretches the elastic posterior capsule of the capsular bag. If the ciliary muscle was not thus maintained in its relaxed state until the completion of fibrosis, the ciliary muscle would undergo essentially normal brain-induced vision accommodation contraction and relaxation during fibrosis. This ciliary muscle action during fibrosis would result in improper formation of the pockets in the fibrosis tissue which contain the extended portions of the lens. Moreover, ciliary muscle contraction during fibrosis would compress the capsular bag and thereby the lens radially in such a way as to very likely dislocate or decenter the lens from its proper position in the bag or fix the optic in the near vision position.

When the cycloplegic effect of the ciliary muscle relaxant wears off after the completion of fibrosis, the ciliary muscle again becomes free to undergo normal brain-induced contraction and relaxation. Normal brain-induced contraction of the muscle then compresses the lens radially, relaxes the zonules and anterior capsule rim, and increases vitreous pressure in the vitreous cavity of the eye. This normal contraction of the ciliary muscle effects anterior accommodation movement of the lens optic for near vision by the combined action of the increased vitreous pressure, anterior capsule rim relaxation, and the anterior bias of the stretched posterior capsule. Similarly, brain-induced relaxation of the ciliary muscle reduces vitreous pressure, relieves radial compression of the lens, and stretches the anterior capsule rim to effect posterior movement of the lens optic for distant vision.

Normal brain-induced contraction and relaxation of the ciliary muscle after the completion of fibrosis thus causes anterior and posterior accommodation axial movement of the lens optic between near and distant vision positions relative to the retina. During this accommodation movement of the optic, the lens extended portions may undergo endwise movement within their pockets in the fibrous tissue.

Thus the optic moves anteriorly and posteriorly relative to the outer ends of the haptics to create a near reading effect. Importantly, it has been discovered that the optic positively bends or bows in order to enhance near and intermediate vision capability. This bending is a result of forward vitreous pressure capsular contraction creating a warpage (that is, bending or bowing) of the optic that enhances the patients' ability to read. This warpage is created by ciliary muscle constriction, resulting in relaxation of the zonules which then allows end-to-end pressure on the optic by fibrosed capsule, creating a positive effect of minus power for the patent.

The described lens embodiments of the invention conform to one of the following basic lens configurations:

A flexible lens body configuration wherein the extending portions and optic are all flexible and the extending portions and optic are in the same plane. This lens after implantation in the eye and after paralyzing the ciliary muscle for two to three weeks, undergoes natural posterior location in the capsular bag space due to end-wise compression and shrink-wrapping of the lens by fibrosis of the anterior capsule.

A lens configuration such that the lens body is flexible throughout the extending portions and optic such that the lens optic before implantation is located behind the outer ends of the extending portions such that the optic can move backwards and forwards along the axis of the eye relative to the outer ends of the haptics. This movement can be such that the optic never moves anteriorly to the outer ends of the extending portions, that it moves from a posterior position to a position which makes it uniplanar to the outer ends of the extending portions, or such that it moves from a posterior position to a position anterior to the outer ends of the extending portions.

An accommodating flexible intraocular lens whereby the extended portions and optic are flexible, wherein the optic is located anteriorly to the outer ends of the extended portions prior to implantation within the eye. The lens is configured such that, with constriction of the ciliary muscle, the optic will move anteriorly relative to the outer ends of the extended portions and posteriorly upon relaxation of the ciliary muscle relative to the outer end of the extended portions.

The optic may or may not move to the same plane as or behind the outer ends of the extended portions.

The three embodiments described above preferably have a reduced thickness portion of the extended portion adjacent to the optic comprising a thinned portion or a groove. Alternatively the extended portions adjacent to the optic may be resiliently flexible without having a hinged or thin portion. Should the material from which the lens is made be relatively rigid, then the whole lens itself may move backwards and forwards without there being any flexion at the optic flexible portion junction.

The movement of the lens alone or the lens optic relative to the outer ends of the extended portions may be caused by one or a combination of the following: constriction and relaxation of the ciliary muscle, increase and decrease of vitreous cavity pressure, the resilience of the posterior capsule, and end-wise compression and relaxation of the lens by the ciliary muscle through the capsular bag wall.

The extended portions of a presently preferred lens embodiment are generally T-shaped haptics each including a haptic plate and a pair of relatively slender resiliently flexible fixation fingers at the outer end of the haptic plate. In their normal unstressed state (e.g., FIG. 2), the two fixation fingers at the outer end of each haptic plate extend laterally outward from opposite edges of the respective haptic plate in the plane of the plate and substantially flush with the radially outer end edge of the plate to form the horizontal “crossbar” of the haptic T-shape. The radially outer end edges of the haptic plates (the fixation fingers) are slightly circularly curved about the central axis of the lens optic to substantially equal radii closely approximating the radius of the interior perimeter of the capsular bag when the ciliary muscle of the eye is relaxed. During implantation of the lens in the bag, the inner perimetrical wall of the bag deflects the haptic fingers generally radially inward from their normal unstressed positions to arcuate bent configurations (FIG. 7) in which the radially outer edges of the fingers and the curved outer end edges of the respective haptic plates conform approximately to a common circular curvature closely approximating the curvature of the inner perimetrical wall of the bag. The outer T-ends of the haptics then press lightly against the perimetrical bag wall and are fixated within the bag perimeter during fibrosis with approximation of the anterior capsule to the posterior capsule to accurately center the implanted lens in the bag with the lens optic aligned with the anterior capsule opening in the bag.

The haptic plates of certain described lens embodiments are narrower in width than the optic diameter and are tapered so as to narrow in width toward their outer ends. These relatively narrow plates of the haptics flex or pivot relatively easily to aid the accommodating action of the lens and form haptic pockets of maximum length in the fibrous tissue between the haptic fingers and the optic which maximize the accommodation movement of the lens optic. The tapered haptics, being wider adjacent to the optic, can slide radially in the capsular bag pockets during contraction of the ciliary muscle to enable forward movement of the optic for vision accommodation.

In a lens embodiment of the invention, the lens optic and extended portions, which preferably are plates, are molded or otherwise fabricated preferably as an integral one piece lens structure in which the inner ends of the extended portions are integrally joined to the optic, and the extended portions have flexible hinges at their inner ends adjacent the optic at which the extended portions are hingable anteriorly and posteriorly relative to the optic. The extended portions are T-shaped haptics formed by embedding flexible haptic fingers at the outer ends of the haptic plates proper.

Accordingly, it is a principal object of the present invention to provide an improved accommodating lens.

### BRIEF DESCRIPTION OF THE DRAWINGS

FIG. 1 is a perspective view of a lens according to the present invention,

FIG. 2 is a plan view thereof,

FIG. 3 is a side view,

FIG. 4 is an end view,

FIG. 5 is a side view similar to FIG. 3 of the lens in FIG. 1 looking in the direction of arrow 5 in FIG. 2 and showing in broken lines the hinging action of the lens haptics,

FIG. 6a is a side view of the optic of the lens, and FIG. 6b is a like side view of the optic but showing an anterior bowing of the optic,

FIG. 7 is a view showing the lens as implanted in the eye,

FIGS. 8 and 9 are alternative embodiments.

FIG. 10 is a section view taken through a human eye having the lens of FIGS. 1 through 4 implanted within a natural capsular bag of the eye, and

FIG. 11 is an enlarged fragmentary section similar to FIG. 10 illustrating the initial placement of the lens in the eye, and FIGS. 12-14 are sections similar to FIG. 11 illustrating the normal vision-accommodating action of the accommodating lens in its axial movement from the posterior position of FIG. 12 to the anterior position of FIG. 14, and

### DESCRIPTION OF PREFERRED EMBODIMENTS

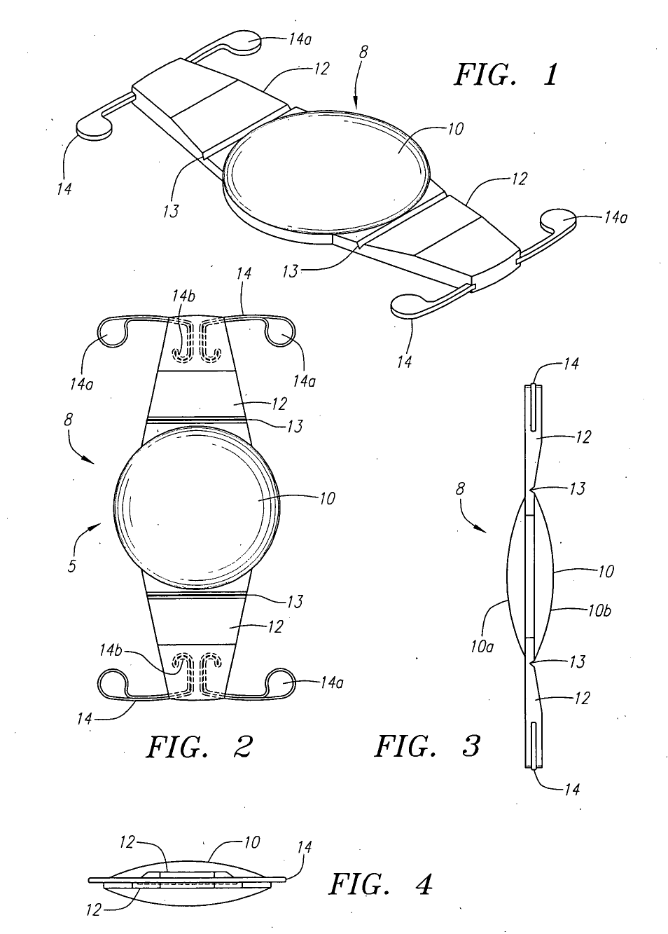

Turning now to FIGS. 1 through 4, an intraocular lens 8 according to the present invention is shown in detail and which replaces and performs the accommodation function of a removed human crystalen lens. The lens 8 may be utilized to replace either a natural lens which is virtually totally defective, such as a cataractious natural lens, or a natural lens that provides satisfactory vision at one distance without the wearing of glasses but provides satisfactory vision at another distance only when glasses are worn. For example, the accommodating intraocular lens of the invention can be utilized to correct refractive errors and restore accommodation for persons in their mid-40s or older who require reading glasses or bifocals for near vision.

Intraocular lens 8 comprises a flexible unitary lens body, including a flexible biconvex solid optic 10, which may be formed of relatively hard material, relatively soft flexible semi-rigid material, or a combination of both hard and soft materials. Examples of relatively hard materials which are suitable for the lens body are methyl methacrylate, polysulfones, and other relatively hard biologically inert optical materials. Examples of suitable relatively soft materials for the lens body are silicone, hydrogels, thermolabile materials, and other flexible semi-rigid biologically inert optical materials.

The lens 8 includes the central optic 10 and T-shaped extended portions or plate haptics extending from diametrically opposite edges of the optic and formed by haptics 12 and fixation fingers 14. The posterior surface 10a (FIG. 3) and the anterior surface 10b may have any suitable curvature such as spherical. The fingers 14 preferably have enlarged ends 14a as best seen in FIGS. 1 and 2.

The haptics include haptic members or plates 12 having inner ends joined to the optic and opposite outer free ends and lateral fixation fingers or loops 14 at their outer ends. The fingers 14 are attached at 14b to the outer ends of the plates 12. The fingers 14 may be of a different but flexible material.

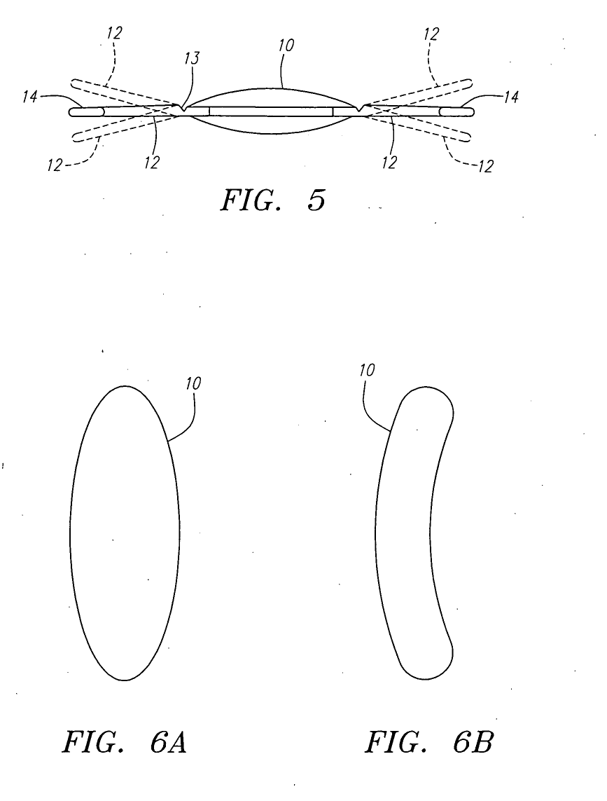

The haptic plates 12 preferably are longitudinally tapered so as to narrow in width toward their outer ends and may have a width throughout their length less than the diameter of the optic 10, and may be resiliently flexible for major portions of their lengths. The optic 10 is movable anteriorly and posteriorly relative to the haptics 12, that is to say the optic is movable anteriorly and posteriorly relative to the outer ends of the haptics. The preferred lens embodiment illustrated is constructed of a resilient semi-rigid material and has flexible hinges 13 which join the inner ends of the haptic plates 12 to the optic 10. The haptics are relatively rigid and are flexible about the hinges anteriorly and posteriorly relative to the optic particularly as shown in FIG. 5. These hinges 13 are formed by grooves (note FIGS. 1 and 3) which can be either on the anterior, posterior, or both sides and extend across the inner ends of the haptic plates 12. In the present preferred embodiment the grooves 13 are in the anterior side 10b as seen in FIG. 3. The haptics 12 are flexible about the hinges 13 in the anterior and posterior directions of the optic. The lens has a relatively flat unstressed configuration, illustrated in FIG. 3 wherein the haptics 12 and their hinges 13 are disposed in a common plane transverse to the optic axis of the optic 10. Deformation of the lens from this normal unstressed configuration by anterior or posterior deflection of the haptics about their hinges creates in the hinges elastic strain energy forces which urge the lens to its normal unstressed configuration.

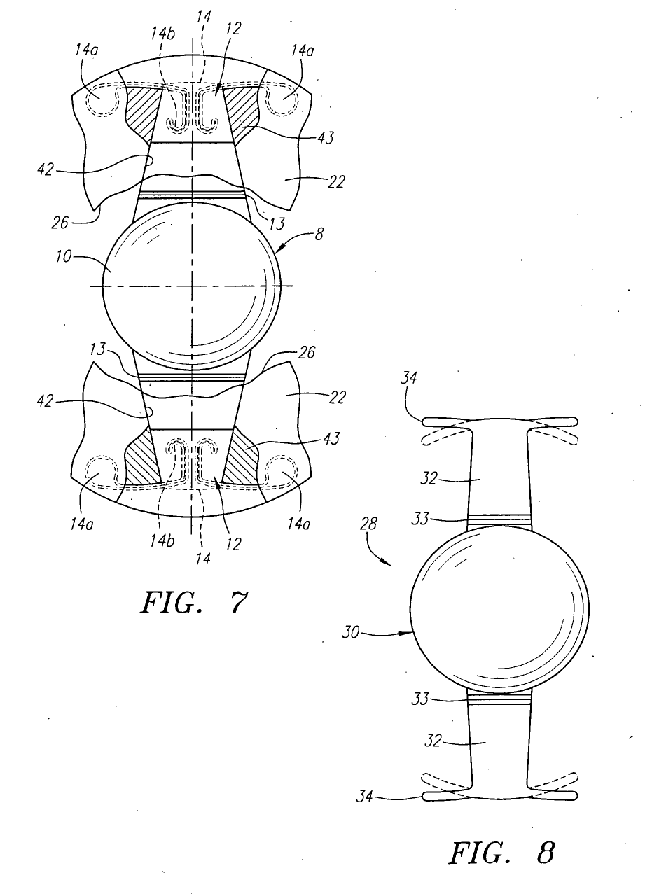

Turning to FIG. 7 the capsular bag (not shown) includes an annular anterior capsular remnant or rim 22. The capsular rim 22 is the remnant of the anterior capsule of the natural lens which remains after capsulorhexis has been performed on the natural lens. This rim circumferentially surrounds the central, generally round anterior opening 26 (capsulotomy) in the capsular bag through which the natural lens matrix was removed from the natural lens. The capsular bag is secured about its perimeter to the cilary muscle via the zonules which are not shown.

In their normal unstressed state shown in solid lines in FIG. 2 the fixation fingers 14 of each plate haptic 12 extend laterally out from opposite longitudinal edges of the respective haptic plate 12 in the plane of the plate and substantially flush with the outer end edge of the plate. When unstressed, the fingers 14 are preferably straight or slightly bowed with a slight radially inward curvature, as shown in FIG. 2. As shown in broken lines in FIG. 7, the fingers 14 are laterally resiliently flexible radially of the haptic plates 12 to their broken line positions of FIG. 7 in which the radially outer edges of the fingers and the end edges of the haptic plates 12 conform substantially to a common circle centered on the axis of the optic 10.

As explained above, it is known that the present accommodating lens moves anteriorly and posteriorly relative to the outer ends of the haptics such as illustrated in FIG. 5 to create a near reading effect. It has been discovered that the optic 10 distorts or bends anteriorly in order to enhance near and intermediate vision capability. FIG. 6a illustrates the optic 10 in its normal state, and FIG. 6b diagrammatically this bending or distortion effect of the optic 10 itself. It is believed that this bending is a result of forward vitreous pressure and capsular contraction creating the bending or warpage of the optic 10 that enhances the ability of the patient to read. This warpage is created by ciliary muscle constriction, resulting in a relaxation of the zonules thus allowing end-to-end pressure on the lens optic by releasing the tension on the optic from the fibrosed capsule, thereby creating a positive effect of minus power for the patient.

Appendix A1-A3 hereto are wavefront analysis showing a bending of the optics 10. These wavefront analyses show in the diagram entitled “Wavefront Total” distance vision, and diagram “Refraction Total” accommodating near vision, and in “Wavefront HOA” the difference or change in power.

As a specific example of the lens of FIGS. 1 through 4, the lens 10 and haptics 12 preferably are silicone and the fingers 14 are polymide. The typical optic 10 has a center thickness of 0.53 to 1.22 mm, but these values can be changed such as increased somewhat, for example, 0.10 mm. The haptic plate typically is 0.45 mm at its thickest, tapering down to 0.25 mm adjacent the optic 10 interface. An exemplary thickness of the fingers is 0.13 mm.

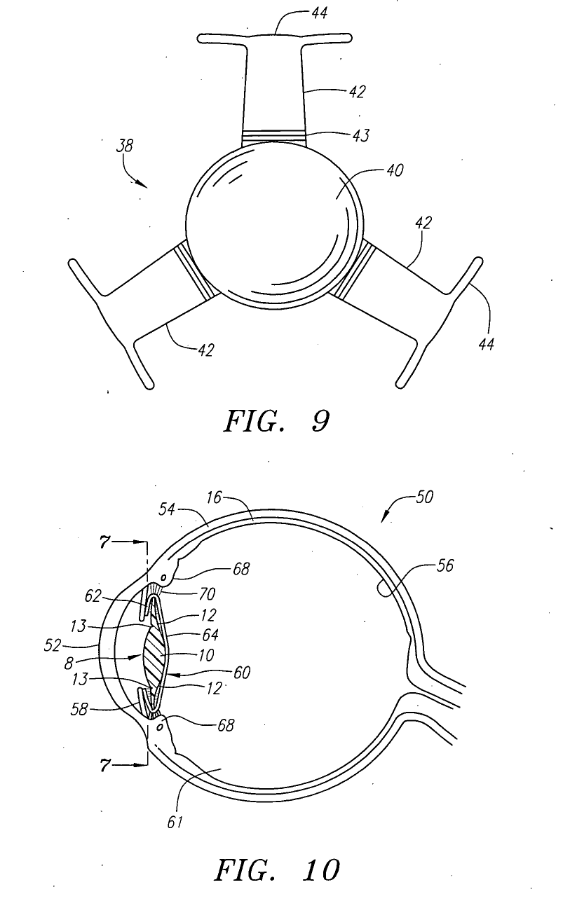

Alternative embodiments are illustrated in FIGS. 8 and 9. The embodiment of FIG. 8 comprises a lens 28 with T-shaped haptics and includes an optic 30, haptics 32, fixation fingers 34 and hinges 33. The lens 38 of FIG. 9 differs in that it has an optic 40 from which extend three haptics 42. The haptics 42 similarly have fixation fingers 44 and they are hinged at 43 to the optic 40.

FIGS. 10 through 14 provide diagrammatic illustrations of the lens of the present invention as implanted in the eye.

Turning first to FIG. 10, there is illustrated a human eye 50 whose natural crystalline lens matrix has been removed from the natural lens capsule of the eye through an anterior opening in the capsule formed by an anterior capsulotomy, in this case a continuous tear circular capsulotomy, or capsulorhexis. As noted earlier, this natural lens matrix, which is normally optically clear, often becomes cloudy and forms a cataract which is cured by removing the matrix and replacing it with an artificial intraocular lens.

Continuous tear circular capsulotomy, or capsulorhexis, involves tearing the anterior capsule along a generally circular tear line in such a way as to form a relatively smooth-edged circular opening in the center of the anterior capsule. The cataract is removed from the natural lens capsule through this opening. After completion of this surgical procedure, the eye includes an optically clear anterior cornea 52, an opaque sclera 54 on the inner side of which is the retina 56 of the eye, an iris 58, a capsular bag 60 behind the iris, and a vitreous cavity 61 behind the capsular bag filled with the gel-like vitreous humor. The capsular bag 60 is the structure of the natural lens of the eye which remains intact within the eye after the continuous tear circular tear capsulorhexis has been performed and the natural lens matrix has been removed from the natural lens.

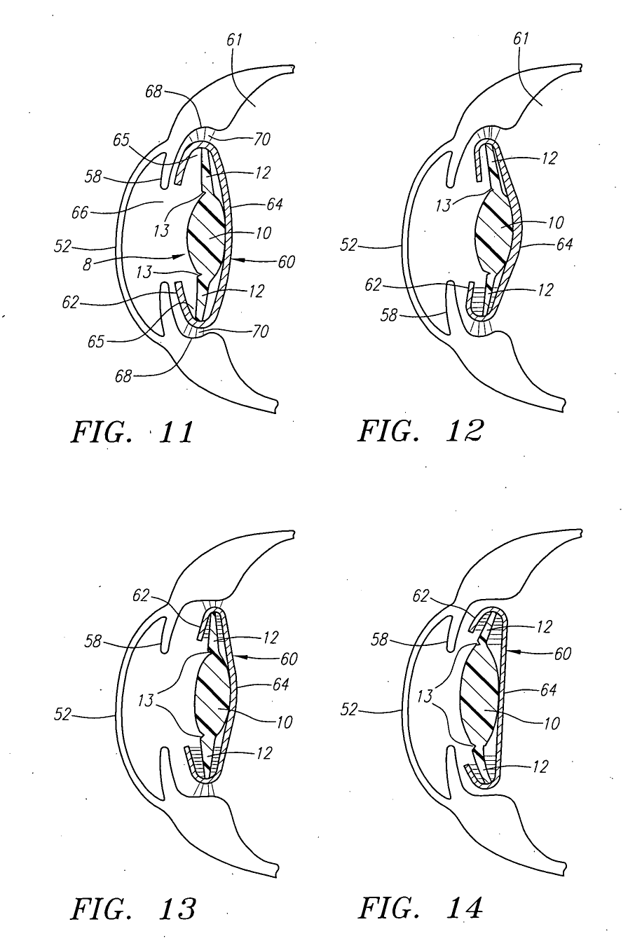

The capsular bag 60 includes an annular anterior capsular remnant or rim 62 and an elastic posterior capsule 64 which are joined along the perimeter of the bag to form an annular crevice-like cul-de-sac 65 (note: FIG. 11) between the rim and posterior capsule. The capsular rim 62 is the remnant of the anterior capsule of the natural lens which remains after capsulorhexis has been performed on the natural lens. This rim circumferentially surrounds a central, generally round anterior opening 66 (capsulotomy) in the capsular bag through which the natural lens matrix was previously removed from the natural lens. The capsular bag 60 is secured about its perimeter to the ciliary muscle 68 of the eye by zonules 70.

Natural accommodation in a normal human eye having a normal human crystalline lens involves automatic contraction or constriction and relaxation of the ciliary muscle of the eye by the brain in response to looking at objects at different distances. Ciliary muscle relaxation, which is the normal state of the muscle, shapes the human crystalline lens for distant vision. Ciliary muscle contraction shapes the human crystalline lens for near vision. The brain-induced change from distant vision to near vision is referred to as accommodation.

Implanted within the capsular bag 60 of the eye 50 is the lens 8 according to this invention which replaces and performs the accommodation function of the removed human crystalline lens. As noted earlier, the accommodating intraocular lens may be utilized to replace either a natural lens which is virtually totally defective, such as a cataractous natural lens, or a natural lens that provides satisfactory vision at one distance without the wearing of glasses but provides satisfactory vision at another distance only when glasses are worn. The lens 8 includes the central optic 10 and T-shaped extended portions or plate haptics 12 extending from diametrically opposite edges of the optic as noted earlier. As shown in broken lines in FIG. 7, the fingers 14 are laterally resiliently flexible radially of the haptic plates 12 to their broken line positions of FIG. 7 in which the radially outer edges of the fingers and the end edges of the haptic plates 12 conform substantially to a common circle centered on the axis of the optic 8.

When implanting the lens in the eye, the ciliary muscle 68 of the eye is paralyzed in its relaxed state, shown in FIG. 11, in which this muscle stretches the capsular bag 60 to its maximum diameter. The lens is inserted into the bag through the anterior capsule opening 66 and is sized in length, endwise of the haptics 12, for placement of the lens in the position shown in FIG. 11. In this position, the lens optic 10 is aligned with anterior opening 66 in the bag, as shown in FIG. 11. The posterior side of the lens faces the elastic posterior capsule 64 of the capsular bag, and the posterior side of the lens optic 10 is disposed in close proximity to or contacts the posterior capsule. The radially outer T-ends of the lens fingers 14 are positioned within the cul-de-sac 65 of the capsular hag with the outer end edges of the haptic plates 12 and the haptic fingers 14 in close proximity to or seating lightly against the capsular bag cul-de-sac wall (as best seen in FIG. 7). This cul-de-sac wall deflects the haptic fingers 14 inwardly to the positions shown in broken lines in FIG. 7. In these deflected positions, the end edges of the haptic plates and the haptic fingers 14 conform closely to the curvature of the cul-de-sac wall to accurately center the lens in the capsular bag. The lens is thus sized and shaped so that when the ciliary muscle 68 is paralyzed in its relaxed state, the lens fits in the capsular bag 60 with a sufficiently close fit to accurately align the lens 10 with the anterior capsule opening 66 in the bag without significantly deforming the bag.

During a post-operative fibrosis and healing period on the order of two to three weeks following surgical implantation of the lens 8 in the capsular bag 60 epithelial cells under the anterior capsular rim 62 of the bag cause fusion of the rim to the posterior capsule 64 by fibrosis. This fibrosis occurs around the lens haptics 12 in such a way that the haptics are “shrink-wrapped” by the capsular bag 60, and the haptics form pockets 42 (FIG. 7) in the fibrosed material 43. These pockets cooperate with the lens haptics to position and center the lens in the eye. In order to ensure proper formation of the haptic pockets 42 and prevent dislocation of the lens by ciliary muscle contraction during fibrosis, sufficient time must he allowed for fibrosis to occur to completion without contraction of the ciliary muscle 68 from its relaxed state of FIG. 11. This is accomplished by introducing a ciliary muscle relaxant (cycloplegic) into the eye before surgery to dilate the pupil and paralyze the ciliary muscle in its relaxed state and having the patient periodically administer cycloplegic drops into the eye during a post-operative period of sufficient duration (two to five weeks) to permit fibrosis to proceed to completion without contraction of the ciliary muscle. The cycloplegic maintains the ciliary muscle 68 in its relaxed state in which the capsular bag 60 is stretched to its maximum diameter (FIG. 11) and immobilized, and the anterior capsular rim 62 is stretched to a taut trampoline-like condition or position. The rim fibroses from this taut condition. The cycloplegic passes through the cornea of the eye into the fluid within the eye and then enters the ciliary muscle from this fluid. While other cycloplegics may be used, atropine is the preferred cycloplegic because of its prolonged paralyzing effect compared to other cycloplegics. One drop of atropine, for example, may last for two weeks. However, to be on the safe side, patients may be advised to place one drop of atropine in the eye every day during the fibrosis period.

The capsular rim 62 shrinks during fibrosis and thereby shrinks the capsular bag 60 slightly in its radial direction. This shrinkage combines with shrink wrapping of the lens haptics 12 produces some opposing endwise compression of the lens which tends to buckle or flex the lens at its hinges 13 and thereby move the lens optic 10 along the axis of the eye. Unless restrained, this flexing of the lens might occur either forwardly or rearwardly. The taut anterior capsular rim 62 pushes rearwardly against the plate haptics and thereby prevents forward flexing of the lens. This fibrosis-induced end-to-end compression of the lens is not sufficient to interfere with proper formation of the haptic pockets in the fibrosed tissue or cause dislocation of the lens. Accordingly, endwise compression of the lens by fibrosis aided by the rearward thrust of the taut capsular rim against the lens haptics 12 causes rearward flexing of the lens from its initial position of FIG. 11 to its position of FIG. 12. The lens haptics 12 are made sufficiently rigid that they will not buckle under the forces of fibrosis. At the conclusion of fibrosis, the lens occupies its posterior position of FIG. 12 wherein the lens presses rearwardly against the elastic posterior capsule 64 and stretches this capsule rearwardly. The posterior capsule then exerts a forward elastic bias force on the lens. This posterior position of the lens is its distant vision position.

Natural accommodation in a normal human eye involves shaping of the natural crystalline lens by automatic contraction and relaxation of the ciliary muscle of the eye by the brain to focus the eye at different distances. Ciliary muscle relaxation shapes the natural lens for distant vision. Ciliary muscle contraction shapes the natural lens for near vision.

The accommodating intraocular lens 8 is uniquely constructed to utilize this same ciliary muscle action, the fibrosed capsular rim 62 the elastic posterior capsule 64, and the vitreous pressure within the vitreous cavity 61 to effect accommodation movement of the lens optic 10 along the optic axis of the eye between its distant vision position of FIG. 11 to its near vision position of FIG. 14. Thus, when looking at a distant scene, the brain relaxes the ciliary muscles 68. Relaxation of the ciliary muscle stretches the capsular bag 60 to its maximum diameter and its fibrosed anterior rim 62 to the taut condition or position discussed above. The taut rim deflects the lens rearwardly to its posterior distant vision position of FIG. 12 in which the elastic posterior capsule 64 is stretched rearwardly relative to the general plane of the fibrosed haptic end portions, by the lens and thereby exerts a forward bias force on the lens. When looking at a near scene, such as a book when reading, the brain constricts or contracts the ciliary muscle. This ciliary muscle contraction has the three-fold effect of increasing the vitreous cavity pressure, relaxing the capsular bag 60 and particularly its fibrosed capsular rim 62, and exerting opposing endwise compression forces on the ends of the lens haptics 12 with resultant endwise compression of the lens. Relaxation of the capsular rim secondary to relaxation of the zonules permits the rim to flex forwardly and thereby enables the combined forward bias force exerted on the lens by the rearwardly stretched posterior capsule and the increased vitreous cavity pressure to push the lens forwardly relative to the general plane of the fibrosed haptic end portions, in an initial accommodation movement from the position of FIG. 11 to the intermediate accommodation position of FIG. 13.

In this intermediate accommodation position, the lens may be substantially flat or uniplanar, and the ends of the lens haptics and their hinges 13 may be disposed substantially in a common plane normal to the axis of the eye. Prior to accommodation, the lens arches rearwardly so that endwise compression of the lens by contraction of the anterior capsule in the fibrosis tends to produce a rearward buckling force on the lens. However, the increased vitreous cavity pressure and the forward bias force of the stretched posterior capsule are sufficient to overcome this opposing rearward buckling force and effect forward accommodation movement of the lens. Although the lens as a whole moves forward, the hinges adjacent to the optic allow the optic to have the maximum potential for forward movement Subsequent brain-induced relaxation of the ciliary muscle 68 in response to looking at a distant scene reduces the vitreous cavity pressure, stretches the capsular bag 60 to its maximum diameter, and restores the anterior capsular rim 62 to its taut trampoline-like condition to effect return of the lens to its distant viewing position of FIG. 12. During accommodation, the lens optic 10 moves along the axis of the eye. The effective power of the optic is selected by the brain to sharply focus incoming light by moving the optic along the axis of the eye by contraction and relaxation of the ciliary muscle.

The lens haptics 12 flex at their hinges 13 with respect to the lens optic 10 during accommodation. Any elastic strain energy forces developed in the hinges during this flexing produces additional anterior and/or posterior forces on the lens. For example, assume that the lens is relatively flat, i.e., that the lens haptics 12 lie in a common plane as shown in FIG. 3 in the normal unstressed state of the lens. In this case, posterior deflection of the lens from its position of FIG. 3 to its distant vision position of FIG. 12 creates elastic strain energy forces in the hinges 13 which urge the lens forwardly back to its unstressed position of FIG. 3 and thus aid the above discussed initial accommodation of the lens in response to contraction of the ciliary muscle. Final accommodation flexing of the lens from its intermediate position of FIG. 13 to its near vision position of FIG. 14 creates elastic strain energy forces in the hinges 13 which urge the lens rearwardly toward its unstressed position should the optic move forward relative to the outer ends of the haptics and thus aid initial return of the lens from its near vision position to its distant vision position in response to relaxation of the ciliary muscle. The lens may be designed to assume some other normal unstressed position, of course, in which case any elastic strain energy forces created in the lens during flexing of the haptics will aid, resist, or both aid and resist accommodation of the lens to its near vision position and return of the lens to its distant vision position depending upon the unstressed position of the lens.

During accommodation, the lens haptic plates 12 slide endwise in their fibrosed tissue pockets. As shown best in FIGS. 1, 2 and 7, the haptics are tapered endwise in width and thickness to enable the haptics to move freely in the pockets. The lens optic 10 moves forwardly toward and rearwardly away from the anterior capsular rim 62.

While embodiments of the present invention have been shown and described, various modifications may be made without departing from the scope of the present invention, and all such modifications and equivalents are intended to be covered.

1. An accommodating intraocular lens wherein the lens comprises a flexible lens body having normally anterior and posterior sides, including a single solid flexible optic,
the lens body having two or more radially extending portions from the optic such that the lens can move anteriorly with contraction of the ciliary body of the eye, and
the lens being sized to be implanted into the capsular bag of the eye such that contraction of the ciliary muscle causes the lens within the capsular bag behind the iris to move forward towards the iris with its contraction and the optic to deform to aid near vision.

2. An accommodating lens according to claim 1 wherein the lens is sized such that the lens is not in contact with the ciliary muscle through the capsular bag wall.

3. An accommodating lens according to claim 1 wherein the lens can move anteriorly and posteriorly.

4. An accommodating lens according to claim 1, wherein the optic can move anteriorly and posteriorly relative to the outer ends of the extending portions.

5. An accommodating lens according to claim 1, wherein internal elastic strain causes the lens to move anteriorly.

6. An accommodating lens according to claim 1, wherein posterior capsule elasticity causes the lens to move anteriorly.

7. An accommodating lens according to claim 1, wherein the optic can move forward and backwards with ciliary muscle contraction and relaxation.

8. An accommodating lens according to claim 7 wherein the optic can move along the axis of the eye relative to the outer ends of the extending portions.

9. An accommodating lens according to claim 1, which is uniplanar.

10. An accommodating lens according to claim 1, which is vaulted forward.

11. An accommodating lens according to claim 1, which is vaulted backwards.

12. An accommodating lens according to claim 1, which is multiplanar.

13. An accommodating lens according to claim 1, which is made from different materials.

14. An accommodating lens according to claim 1, wherein the extending portions are plate haptics.

15. An accommodating lens according to claim 1, wherein the extended portions are plate haptics with hinges.

16. An accommodating lens according to claim 1, wherein the extending portions are plate haptics with a narrowing of the plate junctions adjacent to the optic.

17. An accommodating lens according to claim 1, wherein constriction of the ciliary muscle produces forward movement of the lens optic within the capsular bag towards the iris for near vision.

18. An accommodating lens according to claim 1, wherein the extending portions comprise three spaced plate haptics.

19. An accommodating lens according to claim 1, wherein the extending portions are plate haptics with flexible fingers at their outer ends.

20. An accommodating lens according to claim 1, wherein two or more extending portions comprise plate haptics with a groove across the plate haptic adjacent to the optic.

21. An accommodating lens according to claim 1, wherein the extending portions have lateral fixation fingers having loops.

22. An accommodating lens according to claim 1, wherein the extending portions include hinged plate haptics with laterally extending flexible fixation fingers.

23. An accommodating lens according to claim 1, wherein the lens has extended hinged portions comprising plate haptics which include laterally extending flexible fixation fingers at their outer ends which are made of material different from that of the haptic plates.

24. An accommodating lens according to claim 1, wherein the optic is located posteriorly to the outer ends of the extending portions.

25. An accommodating lens according to claim 1, wherein the optic is biconvex.

26. An accommodating intraocular lens wherein the lens comprises a flexible lens body having normally anterior and posterior sides, including a single solid flexible optic.
the lens comprising the optic and two or more radially extending portions from the optic such that the lens can move anteriorly with contraction of the ciliary body of the eye, the lens and extending portions are formed of silicone with fingers on the outer ends of the extending portions forming together with the extending portions T-shaped haptics, and
the lens being sized to be implanted into the capsular bag of the eye such that contraction of the ciliary muscle causes the lens with the capsular bag behind the iris to move forward towards the iris with its contraction and the optic to be deformed to aid near vision.

27. A lens according to claim 26 wherein the fingers are formed of polyimide.

28. A lens according to claim 26 wherein the optic and extending portions are silicone and the fingers are polyimide.

29. The lens of claim 26,wherein the optic has a center thickness within the range of approximately 0.5 to 1.35, and the extending portions are approximately 0.45 mm thick, and the fingers are approximately 0.13 mm thick.

30. An accommodating intraocular lens wherein the lens comprises a flexible lens body having normally anterior and posterior sides, including a flexible solid silicone optic,
said lens body having at least two radially extending portions from the optic such that the lens can move anteriorly with contraction of the ciliary body of the eye,
the lens being sized to be implanted into the capsular bag of the eye such that contraction of the ciliary muscle can cause the optic within the capsular bag behind the iris to move forward toward the iris and the optic to deform to enhance near and intermediate vision capability as a result of forward vitreous pressure capsular contraction creating a deformity of the optic to enhance a patients' ability to read, this deformity being created by ciliary muscle constriction, creating end-to-end pressure on the capsule lens by the fibrosed anterior and subsequently the intraocular lens, creating a positive effect of minus power for the patient.

31. An accommodating lens according to claim 30, which is uniplanar.

32. An accommodating lens according to claim 30, which is vaulted forward.

33. An accommodating lens according to claim 30, which is vaulted backward.

34. An accommodating lens according to claim 30, wherein the extending portions are plate haptics hinged to the optic.

35. An accommodating lens according to claim 34 wherein the optic and haptics are of silicone.
